(19) United States
(12) Patent Application Publication
(10) Pub. No.: US 2004/0131579 A1
(43) Pub. Date: Jul. 8, 2004

(54) Method and compositions for reducing dermatological aging and for reducing bruising

(75) Inventors: Chaya Duraiswami, Seacaucus, NJ (US); Susan E. Simpson, Wyckoff, NJ (US); Mark S. Garrison, Suffern, NY (US); Dennis M. Martin, Cornwall, NY (US); Roberta C. Bloom, Shelton, CT (US)

Correspondence Address: CHARLES N.J. RUGGIERO, ESQ., OHLANDT, GREELEY, RUGGIERO & PERLE, L.L.P., 10th FLOOR, ONE LANDMARK SQUARE, STAMFORD, CT, 06901-2682, US

(73) Assignee: Avon Products, Inc.

(21) Appl. No.: 10/682,238

(22) Filed: Oct. 9, 2003

Related U.S. Application Data
(63) Continuation of application No. 09/554,004, filed on May 8, 2000, now abandoned.
Application No. 09/554,004 is a 371 of international application No. PCT/US99/20854, filed on Sep. 10, 1999.
(60) Provisional application No. 60/099,698, filed on Sep. 10, 1998.

(51) Int. Cl. A61K 31/7048; A61K 31/353; A61K 7/06; A61K 7/42

(52) U.S. Cl. 424/74; 514/27; 514/456; 424/59

(57) ABSTRACT

There is disclosed a method for reducing susceptibility to, severity or duration of bruising by application of a topical composition. The topical composition has a phytoextract and a vehicle. There is also disclosed a method of improving the aesthetic appearance of skin by application of a topical composition that further contains a secondary ingredient.

### 1. Field of the invention

The present invention relates to topical compositions and methods of using same for improving overall dermatological health. The present invention also relates to compositions for reducing susceptibility to bruising and for decreasing the healing time for bruises that occur and to methods for using these compositions. The present invention further relates to topical compositions to alleviate dermatological symptoms associated with hormonal aging and methods for using same.

### 2. DESCRIPTION OF THE RELATED ART

Aging of the skin results from the synergistic effects of intrinsic aging (due to age and genetic factors), photoaging (due to exposure to ultraviolet radiation), and, for women, hormonal aging (due to estrogen deficiency in peri-menopausal and menopausal women.) Such dermatological aging manifests as skin wrinkles, pigmentation/age spots, sallow skin, sagging skin, thinning of skin, a decrease in skin's elasticity and resilience. One cause of the foregoing manifestations of dermatological aging is a net loss of collagen fibers in skin. This net loss of skin collagen fibers results in thinning of the dermis. Because the dermis acts to “cushion” the force of impact, this decrease in the thickness of the dermis can result in increased bruising.

In addition, some women also experience an increase in acne because of these hormonal changes.

A need exists for a composition and method that will retard or reverse the negative dermatological effects associated with hormonal aging. A further need exists for a composition and method that will accomplish the foregoing with minimal adverse effects.

PCT publication WO 98/21946 to Wurtman et al. (the disclosure of—-which is incorporated herein by reference) provides dietary supplements that include phytoestrogen compounds, and either (a) remedial carbohydrates, (b) choline compounds or (c) both.

PCT publication WO 98156373 to Gorbach (the disclosure of which is incorporated by reference herein) provides topical compositions that utilize purified isoflavonoids, such as genistein, daidzein, biochanin A, formononetin, O-desmethylangolensin, glycitin, and equol. Gorbach also provides topical compositions having between 1 and 40 mg of purified isoflavones per gram of base (i.e. 0.1 wt % to 4 wt %) for treating and preventing wrinkles. Gorbach defines “purified isoflavonoid” as an isoflavonoid in a more concentrated form than occurs in plants.

U.S. Pat. No. 5,166,132 to Gordon provides topical compositions having an improved enzyme-modified casein solution that are useful for healing and relieving bruising.

### BRIEF SUMMARY OF THE INVENTION

It is an object of the present invention to provide topical compositions and methods to reduce dermatological aging.

It is another object of the present invention to provide topical compositions and methods to reduce susceptibility to bruising.

It is further an object of the present invention to provide topical compositions and methods to reduce the severity of and healing period for bruises that do manifest.

### DETAILED DESCRIPTION OF THE INVENTION

A “topical composition” as used herein refers to a composition intended to be directly applied or spread on the surface of skin. An “effective amount” means an amount of a compound or a composition sufficient to induce a positive change in the skin condition. A “physiologically acceptable vehicle” or a “suitable topical vehicle” refers to a drug, cosmetic, medicament or inert ingredient that is suitable for use in direct contact with human tissues without undue toxicity. All percentages refer to weight percent, based on the total weight of the topical composition.

The first principal component of the present compositions is a phytoextract. The phytoextract is preferably a phytoestrogen and, more preferably, either a (1) isoflavone, (2) steroidal, (3) sterol, (4) coumestan, (5) lignan, or (6) any mixture thereof.

The term “phytoextract” as used herein encompasses all such compounds that occur naturally in plants regardless of whether the actual compound used in the present invention is extracted from plant sources or manmade. As stated above, it is preferred that the phytoextract is a phytoestrogen. Preferably, the phytoextract is derived from plant sources In the more preferred embodiment, the phytoextract is a phytoestrogen that is naturally derived. Alternatively, the phytoextract may be synthetically derived. “Phytoestrogen” as used herein refers to compounds that are either (a) known to exhibit an estrogen-type effect or (b) specifically set forth herein as a phytoestrogen.

Exemplary phytoextracts and their sources are set forth below in Table 1.

TABLE 1

| Phytoextract Active | Source |
|---|---|
| Segetalins B, C, and D | Vaccaria segetalis. |
| Isoflavone 6-Prenylisocaviunin | Sopubia delphinifolia |
| 3′-Prenyl-4′methoxy-isoflavone-7-O-β-D- | |
| (2′O-p-coumaroyl glucopyranoside) | |
| Kaempferol, | Fennel |
| β-Sitosterol, | (Foeniculum vulgare) |
| Cimifugin or Macrotin, | Black Cohosh |
| Cimigenol or Cimifugol, | (Cimifuga racemosa) |
| Cimigoside or Cimigenol | |
| Xyloside, Formononetin | |
| Isoferulic acid, Cimifugoside | |
| Daidzein, Genistein, Equol | Soy |
| | (Glycine Max) |
| Liqcoumarin, | Licorice |
| 6-Acetyl-5-hydroxy-4-methylcoumarin, | (Glycyrrhiza glabra) |
| Formononetin, Glabridin, Hispaglabridin-A, | |
| Hispaglabridin-B, Isoliquiritigenin | |
| Genistin, Biochanin A, | Alfalfa |
| Coumesterol, Formononetin | (Medicago sativa) |
| β-Sitosterol, | Bourbon |
| Biochanin-A | |
| β-sitosterol, | Beer |
| Genistein, | |
| Daidzein | |
| β-Sitosterol, | Saw Palmetto |
| Naringenin | Eucalyptus, |
| | Citrus Fruits, e.g. |
| | Oranges, Lemons |
| Humulone, | Hops |
| Xanthohumol, | |
| Lactaric acid | |
| Coumestans, | Sunflower |
| 25-Dehydro-sitostanol, | |
| 23-Dehydro-β-sitosterol, | |
| Phytosterols | |
| Estradiol, Estrone | Pomegranate |
| Phytosterol | |
| β-Sitosterol | |
| Phytosterol, Coumesterol, Indole-3- | Brussel sprouts |
| Carbinol, Quercitin | |
| Diosgenin, DHEA | Yam |
| | (Dioscorea Species) |
| Biochanin A, Formononetin, Genistin, | Red Clover |
| Coumesterol | (Trifolium pratense) |
| Matairesinol | Palm |
| Enterodiol, Enterolactone | |
| Brassicasterol, Campesterol, β-Sitosterol, | Canola |
| Stigmasterol | |
| Zearalenone sulfate | Fusarium species |
| Biochanin A | Legumes |
| Furanoid Lignans | Chaparral |
| | (Larrea tridentata) |
| Matairesinol | Rye |

The more preferred phytoextracts are phytoestrogens, such as daidz in, daidzin, acetyl daidzin, malonyl daidzin, glycitin, acetyl glycitin, malonyl glycitin, glycitein, genistin, acetyl genistin, malonyl genistin, genistein, equol, and any mixture thereof. The most preferred phytoextracts are daidzein, glycitein, genistein, equol and any mixture thereof. When the phytoextract is daidzein, glycitein, genistein, equol, and any mixture thereof, the topical composition preferably comprises from about 0.01 wt % to about 0.1 wt % phytoextract, more preferably from about 0.015 wt % to about 0.08 wt % phytoextract, and most preferably from about 0.02 wt % to 0.072 wt % phytoextract.

Examples of preferred plant sources include soy, soy bean, red clover, pomegranate, saw palmetto, canola, and any mixture thereof.

In addition to the phytoextract component, the present invention preferably includes a secondary component. The secondary component is selected from one or more of the following twelve groups.

1. An estrogen synthetase stimulating compound: Examples of such a compound include caffeine and/or derivatives thereof, and any mixture thereof. Caffeine is the more preferred of such compounds.

2. A compound capable of inhibiting 5 alpha-reductase activity: Examples of such a compound include linolenic acid, linoleic acid, finasteride, and any mixture thereof.

3. An exfoliation promoting compound: Suitable examples include alpha hydroxy acids; beta hydroxy acids; oxa acids as disclosed in U.S. Pat. No. 5,847,003 (the disclosure of which is incorporated by reference herein); oxa diacids as disclosed in U.S. Pat. No. 5,834,513 (the disclosure of which is incorporated by reference herein); mechanical exfoliation compounds, such as bamboo exfoliant extract; salicylic acid; benzoyl peroxide; alpha-keto acids, such as pyruvic acid, 2-oxopropanoic acid, 2-oxobutanoic acid, and 2-oxopentanoic acid; and any mixture thereof.

The preferred exfoliation promoting compounds are lactic acid, glycolic acid, 3,6,9-trioxaundecanedioic acid, and any mixture thereof. When present invention includes an exfoliation promoting compound, the composition comprises about 1 wt % to 20 wt %, preferably 1 wt % to about 15 wt %, more preferably about 4 wt % to about 10 wt % acid, and most preferably about 4 wt % of the exfoliation promoting compound.

4. An ultraviolet (UV) light protecting/sunscreen agent: Examples include organic and inorganic sunscreens, such as titanium dioxide, zinc oxide, methyl benzylidene camphor and/or its derivatives, octocrylene, anthranilates, benzophenones, butylmethoxydibenzoylmethane (avobenzone), naphtholsulphonates, benzoic acid derivatives, salicylates, cinnamic acid derivatives, and mixtures thereof. Of these, butylmethoxydibenzoylmethane (PARSOL 1789), octocrylene, octylsalicylate, octylmethoxycinnamate and oxybenzone and mixtures thereof are preferred. Butylmethoxydibenzoylmethane or avobenzone (PARSOL 1789), oxybenzone and octylmethoxycinnamate, and mixtures thereof are most preferred. In addition, U.S. Pat. No. 5,824,702, to Wei (the disclosure of which is incorporated by reference herein) provides that genistein exhibits protective activity against ultraviolet induced skin photodamage and cancer. Co-formulation with an ultraviolet light protecting/sunscreen agent is particularly desirable when the present invention is prepared for consumers who engage in outdoor activities.

5. Retinoids: Examples of suitable retinoids include retinol, retinoic acid, retinyl palmitate, retinyl propionate, retinyl acetate, isotretinoin as well as synthetic retinoid mimics, and derivatives of the foregoing.

6. Hirsutism inhibiting agents: Examples of such agents include γ-linolenic acid, linoleic acid, and derivatives thereof.

7. Barrier function enhancing agents: Examples include ceramides, essential fatty acids and their esters, especially glycerides, α-hydroxy fatty acids and their esters derived with alkanols through carboxylic hydroxyl or with other fatty acids at the omega-hydroxyl, the latter type being most preferred, with phospholipids, cholesterol and its esters, such as cholesteryl hemisuccinate and cholesteryl phosphate of which cholesterol phosphate and essential fatty acids are most preferred, cholestanol and its derivatives. The barrier function enhancing agent can be added to a topical composition either as singular molecular entities or as a complex mixture of lipids derived from either synthetic, animal or plant sources.

8. Collagen enhancing agents: These agents prevent skin sagging by promoting a net increase in collagen, either by reducing collagen breakdown or by promoting collagen formation. Examples of such inhibitors include Clara extract (Sophora augustifolia), ascorbyl-phoshoryl-cholesterol, ascorbic acid, ascorbic acid derivatives, and any mixtures thereof.

9. Elastase inhibitors: Examples of these inhibitors include Honeysuckle extract (Lonicera caprifolium). These inhibitors act to prevent sagging of the skin.

10. Skin lightening agents: Examples include kojic acid hydroquinone, licorice derivatives, ascorbic acid/ascorbic acid derivatives (e.g. magnesium ascorbyl phosphate), arbutin, bearberry (Arctostaphylos uva ursi), Glycyrrhiza glabra and its derivatives, Chlorelia vulgaris extract, and—any mixture thereof.

11. Antioxidants: Examples include compounds having phenolic hydroxy functions, such as ascorbic acid, ascorbic acid derivatives, gallic acid derivatives (e.g. propyl gallate); ferulic acid derivatives (e.g. ethyl ferulate, sodium ferulate); nitrones; N-tertbutyl-nitrone; I-(4-pyridyl-1-oxide)-N-tertbutyl-nitrone; curcumin, tetrahydrocurcumin; 6-hydroxy-2,5,7,tetramethylchroman-2-carboxylic acid; uric acid; reductic acid; tannic acid; rosmarinic acid; tocopherol and its derivatives; catechins; and any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as sulfites, bisulfites, metabisulfite, or other inorganic salts and acids containing sulfur.

12. Skin cooling compounds: Examples include menthol, menthyl glycerol, asymmetrical carbonates, thiocarbonates and urethanes, N-substituted carboxamides, ureas or phosphine oxides, menthyl lactate, menthone glycerine acetal, and any mixtures thereof. Since many women experience “hot flashes/flushes” during perimenopause, coformulation with skin cooling compounds is particularly desirable when providing topical compositions of the present invention for such women.

The secondary component enhances the dermatological benefits achieved by the phytoextract. It is more preferred that the compositions of the present invention include at least two secondary components with each secondary component being selected from a different group. Table 2, below, sets forth examples of such preferred combinations.

TABLE 2
Examples of combinations of phytoestrogen with
secondary component.

| | Formula Number | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Phytoestrogen | X | X | X | X | X | X | X | X | X | X | X | X |
| Glycolic Acid | X | X | X | | | | | X | | | | X |
| Lactic Acid | | | | X | | | | | X | | | |
| Oxa Acid | | | | | | X | | | | | | X |
| Retinol | | | | X | X | | | | | X | X | X |
| Ascorbyl- | | | | | | | X | X | | | | X |
| phosphoryl- | | | | | | | | | | | | |
| cholesterol | | | | | | | | | | | | |
| Ascorbic Acid | X | | | | | | | | X | X | | X |
| Octylmeth- | X | X | | X | X | X | X | X | X | X | X | X |
| oxycinnamate | | | | | | | | | | | | |
| Oxybenzone | X | X | | X | X | X | X | X | X | X | X | X |
| Avobenzone | X | X | | X | X | X | X | X | X | | | X |
| Licorice root | | X | X | | | | | | | | | X |
| Tetrahydro- | | | X | | | | | | | | | X |
| curcumin | | | | | | | | | | | | |
| Nitric Oxide | | | | | | | | | | | X | X |
| Synthase | | | | | | | | | | | | |
| Inhibitor | | | | | | | | | | | | |

The compositions of the present invention can include other cosmetic and pharmaceutical actives and excipients. Such suitable cosmetic and pharmaceutical agents include, but are not limited to, antifungals, vitamins, anti-inflammatory agents, antimicrobials, analgesics, nitric oxide synthase inhibitors, insect repellents, self-tanning agents, surfactants, moisturizers, stabilizers, preservatives, antiseptics, thickeners, lubricants, humectants, chelating agents, skin penetration enhancers, emollients, fragrances and colorants.

Examples of suitable thickening agents include xanthan gum, hydroxypropyl cellulose, hydroxyethyl cellulose, carbomer, gum acacia, Seppigel 305 (available from Seppic Co., France), and magnesium aluminum silicate.

The topical compositions of the present invention can include, and their utility can be enhanced by, humectants, such as urea, pyrrolidone carboxylic acid, amino acids, sodium hyaluronate, certain polyols and other compounds with hygroscopic properties.

Topical compositions of the present invention can also include one or more of the following:

(i) vitamins, such as any B vitamin; 1,25-dihydroxy vitamin D3; vitamin K; tocopherol and its derivatives; tocotrienols and their derivatives; nicotinic acid and its esters; pantothenic acid and it esters; panthenol; folic acid and its derivatives; phytic acid; ascorbic acid and its derivatives; vitamin A and its derivatives; and any mixtures thereof;

(ii) antifungals, such as tolnaftate and ketoconazole;

(iii) self-tanning agents, such as dihydroxyacetone and lawsone;

(iv) anti-microbial agents, such as erythromycin and tetracycline;

(v) topical analgesics, such as lidocaine, benzocaine, butacaine, tetracaine, clove oil and eugenol;

(vi) anti-inflammatory agents may be included in topical compositions of the present invention. These anti-inflammatory agents are used at concentrations from about 0.025 wt % to about 10 wt %, preferably, about 0.5 wt % to about 1 wt %, with the concentration of the anti-inflammatory adjusted upward or downward depending upon the pot ncy—of the utilized agents. Examples indude hydrocortisone, prednisone, prednisolone, aspirin, aspirin derivatives, aloe vera, willow bark, chamomile, and mixtures thereof;

(vii) nitric oxide synthase inhibitors that reduce skin redness, vasodilation and inflammatory reactions, especially in response to electromagnetic and ionizing radiation or to the action of chemically or biochemically aggresive compounds can be included in the present invention. The nitric oxide synthase inhibitors are more preferably selected from the group including guanidine derivatives, especially monoaminoguanidine and methylguanidine, L-arginine derivatives, especially NG-nitro-L-arginine and its esters, NG-monomethyl-L-arginine, 2-iminopiperidines, asymmetric dimethyl arginine (ADMA), boronic acid analog of L-arginine (Boroarg-OH.2HCl), and other 2-iminoazaheterocycles;

(viii) insect repellents, such as aliphatic, cyclic or aromatic amides, citronella oil, terpineol, cineole, neem oil, terephthalic acid and its esters, ethyl butylacetylaminopropionate and N,N-diethyl-m-toluamide (DEET) may be included in the present invention. Coformulation with insect repellents can be particularly desirable when the present invention is used to prevent bruising. For those who engage in vigorous outdoor activities, such as hiking and other sports, having an insect repellent incorporated into the present invention provides a convenient two-in-one preventative measure.

The vehicle can comprise most conventional emollients including mineral oil, petrolatum, paraffin, ceresin, ozokerite, microcrystralline wax, perhydrosqualene dimethyl polysiloxanes, methylphenyl polysiloxanes, silicone-glycol copolymers, triglyceride esters, acetylated monoglycerides, ethoxylated glycerides, alkyl esters of fatty acids, fatty acids and alcohols, lanolin, lanolin derivatives, polyhydric alcohol sters, sterols, beeswax derivatives, polyhydric alcohols and polyethers, and amides of fatty acids.

The emulsifiers can be cationic, anionic, nonionic, amphoteric, or a combination thereof. Nonionic emulsifiers are preferred. Exemplary nonionic emulsifiers are commercially available sorbitans, alkoxylated fatty alcohols and alkyl polyglycosides. Anionic emulsifiers may include soaps, alkyl sulfates, monoalkyl and dialkyl phosphates, alkyl sulphonates and acyl isothionates.

The preservatives suitable for use with the present compositions include alkanols, especially ethanol and benzyl alcohol, parabens, sorbates, benzoic acid/benzoates, urea derivatves, and isothiazolinones.

The general activity and mildness to skin of the present topical compositions can also be enhanced by neutralization to pH about 3.5 to about 7.0, most preferably from pH about 3.7 to about 5.6, with one or more of ammonium hydroxide, potassium hydroxide, sodium hydroxide, arginine or other amino acids, and/or triethanolamine.

The utility of the present topical compositions can also be enhanced by certain chelating agents incorporated into the composition at levels from about 0.01% to about 25% by weight, more preferably from about 0.5% to 10%, and most preferably from about 1% to about 5%. Suitable examples of chelating agents include those that have a high affinity for zinc, calcium, magnesium, iron and/or copper ions, such as ethylene-diamine-tetra-acetic acid.

The topical compositions of the present invention can be further formulated according to procedures known in the art to provide cosmetic compositions such as emulsions, gels, creams, lotions, ointments, pastes, sticks, cakes, pencils, essences and serums as well as other topical cosmetic vehicles. It is also contemplated that topical compositions of the—present invention can be incorporated into delivery systems such as liposomes and topical patches, tapes, and sprays.

The topical compositions of the present invention are useful to improve the aesthetic appearance of skin by any one of the following methods:

1. Reducing dermatological aging, particularly dermatological aging due to hormonal aging;

2. Decreasing skin fragility;

3. Preventing and reversing loss of collagen;

4. Preventing skin atrophy;

5. Promoting/accelerating cell turnover;

6. Providing cushion for blood vessels;

7. Improving skin texture;

8. Decreasing fine lines and wrinkles;

9. Improving skin tone;

10. Enhancing skin thickness;

11. Decreasing pore size;

12. Minimizing skin discoloration;

13. Restoring skin luster;

14. Minimizing signs of fatigue;

15. Reducing acne;

16. Decreasing susceptibility to bruising; and

17. Decreasing severity of bruising;

18. Decreasing time required for healing of bruises.

The present invention also includes methods by which these compounds can be used to address the aforementioned skin conditions. Such methods include topically applying an effective amount of the composition of the present invention to areas of the skin, typically once or twice daily. When the present invention is used to improve the overall aesthetic appearance of skin, the preferred areas of application include the face and neck areas. When the present invention is used to reduce susceptibility, severity and healing time of bruises, preferred areas of application include hands, arms (particularly, forearms), legs (particularly, lower leg areas), hips and any other areas subject to impact.

### EXAMPLE 1

Thirty-seven women participated in a twelve week clinical study to evaluate the efficacy of a topical composition, Sample A, in increasing the thickness of the skin. The women treated one forearm with a test product for twelve weeks. The arm to be treated (right or left) was assigned randomly as the test area. Treatment was once every morning and sample A was applied to the test area.

### Sample A

| Ingredients | wt % |
|---|---|
| lactic acid (85%) | 4.71 |
| soy extract (0.08%) | 25.00 |
| vehicle | qs |

The skin was then assessed visually at four weeks and at eight weeks. Visual improvements of the treated arm were very evident. At four weeks, the skin looked and felt smoother. The skin also appeared brighter. At eight weeks, pigment discolorations looked lighter, and the skin had a much better overall appearance. If the treated skin was laterally compressed, the treated skin was far more resistant to compression. Untreated skin compressed easily, resulting in visible crinkling.

It is believed that the resistance to compression exhibited by the treated skin was the result of the epidermis and dermis becoming “plumper” or thicker. The resulting increase in the thickness is believed to be result of activity of the lactic acid, the soy extract, or the combination of the two materials.

### EXAMPLE 2

Since thicker skin provides more “cushion,” which might result in less of a bruise from an impact or a pinch, the following twelve week study was conducted to determine the efficacy of the present invention to reduce the susceptibility of the skin to bruising.

The test utilized Sample A set forth above. Fifteen of the thirty-seven women who participated in the test discussed in Example 1, above, agreed to have their arms bruised at the twelve-week time point. All of these women were post-menopausal.

The volar forearm was bruised using a pinch method. The skin was given a calibrated pinch using Lange Skinfold Calipers. Both treated and untreated volar forearms were pinched approximately two inches from the elbow flex region.

The women returned to the laboratory at one day, four days, and seven days after bruising. Minolta chromameter L-a-b measurements (an objective measurement of skin color) were taken prior to bruising and on the post-bruising follow-up days. Each woman also completed a self-assessment questionnaire on the evaluation days.

Four days post-bruising was selected as the best day for bruise assessments. Not all bruises appeared the first day after bruising, but all bruises appeared within four days of trauma. Furthermore, of those bruises that appeared early (one day post-trauma) some had completely resolved seven days later. Therefore, day four post-bruising was selected as the best time point to assess bruises.

L-a-b Color Measurements of Bruises

Chromameter measurements done in the L-a-b mode assessed three color components of the bruise color. The “L” value is an assessment of light/dark. The “a” value is an assessment of red/green. The “b” value is an assessment of yellow/blue. The following are the average change from baseline (normal skin color) values obtained for “L”, “a” and “b”.

Change from Baseline Values - 4 Days Post-Bruising

| | Treated | Untreated |
|---|---|---|
| L (Light) | −1.1 | −2.9 |
| a (red) | 0.26 | 2.02 |
| b (yellow) | 1.7 | 2.8 |

The higher the “L” value the lighter the skin. Therefore, the untreated bruised skin was darker (or more bruised) than the treated bruised skin. The “a” value increases with greater redness. Untreated bruises were redder than treated bruises. The yellow component of the bruise, the “b” value, is more yellow with a greater number. The “b” value can also be a blue measurement, however, most of the values obtained were in the yellow range (positive) of the scale. The average “b” value for untreated skin was greater for untreated skin than treated skin.

The majority of the panelists had less bruising on the treated arm. The following chart shows the number of panelists with better “L” (lighter), “a” (less red) or “b” (less yellow) values for the treated or untreated arm:

Number of Panelist with Better Values - Day 4 Post-Bruising

| | Treated | Untreated |
|---|---|---|
| Lighter (L) | 11 | 4 |
| Less Red (a) | 10 | 5 |
| Less Yellow (b) | 11 | 4 |

Tables with individual data for “L”, (Table 3), “a” (Table 4), and “b” (Table 5) are set forth below.

TABLE 3
Change from Baseline in “L” Value
(Bruise Darkness) - Day 4 Post-Bruising

| | | Untreated | |
|---|---|---|---|
| Subject # | Treated Arm | Arm | Which Better |
| 1 | −6.9 | −5.5 | U |
| 2 | −1.6 | −3.9 | T |
| 3 | 0.2 | −9.4 | T |
| 4 | −1.3 | 1.5 | U |
| 5 | −0.4 | −1.3 | T |
| 6 | −3.5 | −9.4 | T |
| 7 | 2.2 | 3.4 | U |
| 8 | 0.7 | −2.6 | T |
| 9 | −2.5 | −3.3 | T |
| 10 | 0.4 | −4.1 | T |
| 11 | −3.9 | −3.7 | U |
| 12 | 0.2 | −0.2 | T |
| 13 | 0.8 | −2.0 | T |
| 14 | 0.7 | −1.0 | T |
| 15 | −0.7 | −3.3 | T |
| AVERAGE | −1.1 | −2.9 | |

Note:
The higher the “L” value, the lighter the skin.

TABLE 4
Change from Baseline in “a” Value 4 Days Post Bruising

| | | Untreated | |
|---|---|---|---|
| Subject # | Treated Arm | Arm | Which Better |
| 1 | 3.5 | 3.3 | U |
| 2 | 1.0 | 3.3 | T |
| 3 | −0.2 | 5.9 | T |
| 4 | −0.3 | −0.9 | U |
| 5 | 1.0 | 0.3 | U |
| 6 | −1.3 | 5.2 | T |
| 7 | −2.0 | −2.8 | U |
| 8 | 0.3 | 2.7 | T |
| 9 | 1.0 | 0.9 | U |
| 10 | −0.2 | 2.4 | T |
| 11 | 1.5 | 4.6 | T |
| 12 | −0.7 | 0.2 | T |
| 13 | 0.2 | 2.2 | T |
| 14 | −0.8 | 1.3 | T |
| 15 | 0.9 | 1.7 | T |
| AVERAGE | 0.26 | 2.02 | |

Note:
A higher “a” value indicates skin is redder.

TABLE 5
Change from Baseline in “b” Value 4 Days Post Bruising

| | | Untreated | |
|---|---|---|---|
| Subject # | Treated Arm | Arm | Which Better |
| 1 | 5.9 | 6.5 | T |
| 2 | 1.9 | 3.9 | T |
| 3 | 0.4 | 5.2 | T |
| 4 | 2.6 | 2.9 | T |
| 5 | 0.1 | 0.0 | U |
| 6 | 5.5 | 3.9 | U |
| 7 | −0.3 | −1.8 | U |
| 8 | 0.4 | 3.6 | T |
| 9 | 0.4 | 2.5 | T |
| 10 | 2.4 | 5.6 | T |
| 11 | 6.8 | 0.6 | U |
| 12 | 1.0 | 2.0 | T |
| 13 | −0.8 | 1.1 | T |
| 14 | −1.3 | 2.7 | T |
| 15 | 0.5 | 3.1 | T |
| AVERAGE | 1.7 | 2.8 | |

Note:
Higher “b” value indicates skin is more yellow.

The data clearly suggest that daily use of a product, such as Sample A, can help reduce the susceptibility to bruising. Bruising may still occur, but bruises may be less severe.

Various modifications and alterations to the present invention may be appreciated based on a review of this application. These changes and additions are intended to be within the scope and the spirit of the present invention as defined by the following claims.

What is claim d is:

1. A method for reducing susceptibility to, severity or duration of bruising comprising applying a topical composition comprising a phytoextract and a vehicle.

2. The method of claim 1, wherein the phytoextract is a phytoestrogen.

3. The method of claim 2, wherein the phytoestrogen is selected from the group consisting of: isoflavones, steroidals, sterols, coumestans, lignans, and any mixture thereof.

4. The method of claim 1, wherein the phytoextract is selected from the group consisting of: daidzein, daidzin, acetyl daidzin, malonyl daidzin, glycitin, acetyl glycitin, malonyl glycitin, glycitein, genistin, acetyl genistin, malonyl genistin, genistein, equol, and any mixture thereof.

5. The method of claim 1, wherein the phytoextract is from about 0.01 wt % to about 0.1 wt % of the topical composition.

6. The method of claim 1, wherein the topical composition further comprises a secondary component selected from the group consisting of:
(i) an estrogen synthetase stimulating compound;
(ii) a 5 alpha-reductase activity inhibiting compound;
(iii) an exfoliation-promoting compound;
(iv) an ultraviolet (UV) light protecting/sunscreen agent;
(v) a retinoid;
(vi) a hirsutism inhibiting agent;
(vii) a barrier function enhancing agent;
(viii) a collagen enhancing agent;
(ix) an elastase inhibitor;
(x) a skin lightening agent
(xi) an antioxidant;
(xii) a skin cooling agent; and
(xiii) any mixtures thereof.

7. The method of claim of claim 6, wherein the secondary component is the exfoliation-promoting compound selected from the group consisting of alpha-hydroxy acids, p-hydroxy acids, keto acids, oxa acids, oxa diacids, derivatives thereof, and any mixture thereof.

8. The method of claim 6, wherein the exfoliation-promoting compound is selected from the group consisting of: lactic acid; glycolic acid; 3,6,9-trioxaundecanedioic acid, and any mixtures thereof.

9. The method of claim 6, wherein the topical composition comprises two or more secondary components and each of said secondary components is selected from a different group of said secondary components.

10. The method of claim 6, wherein the topical composition further comprises a tertiary component selected from the group consisting of: antifungals, vitamins, anti-inflammatory agents, antimicrobials, analgesics, nitric oxide synthase inhibitors, insect repellents, self-tanning agents, surfactants, moisturizers, stabilizers, preservatives, antiseptics, thickeners, lubricants, humectants, chelating agents, skin penetration enhancers, emollients, fragrances, colorants, and any mixture thereof.

11. The method of claim 1, wherein the method includes applying topical composition twice daily.

12. The method of claim 1, wherein the vehicle is selected from the group consisting of: solid, solution, essence, serum, pencil, spray, lotion, emulsion, cream, micro-emulsion, gel, ointment, patch, tape, and powder.

13. A method for reducing susceptibility to, severity or duration of bruising in perimenopausal or menopausal women comprising applying a topical composition comprising a phytoestrogen and a vehicle.

14. A method of improving the aesthetic appearance of skin comprising topically applying a composition comprising a phytoestrogen and a vehicle.

15. The method of claim 14, wherein the phytoestrogen comprises from about 0.01 wt % to about 0.08 wt % of the topical composition.

16. The method of claim 15, wherein the phytoestrogen is selected from the group consisting of: daidzein, daidzin, acetyl daidzin, malonyl daidzin, glycitin, acetyl glycitin, malonyl glycitin, glycitein, genistin, acetyl genistin, malonyl genistin, genistein, equol, and any mixture thereof.

17. The method of claim 14, wherein the topical composition further comprises a secondary ingredient selected from the group consisting of:
(i) an estrogen synthetase stimulating compound;
(ii) a 5 alpha-reductase activity inhibiting compound;
(iii) an exfoliation-promoting compound;
(iv) an ultraviolet (UV) light protecting/sunscreen agent;
(v) a retinoid;
(vi) a hirsutism inhibiting agent;
(vii) a barrier function enhancing agent;
(viii) a collagen enhancing agent;
(ix) an elastase inhibitor;
(x) a skin lightening agent;
(xi) an antioxidant;
(xii) a skin cooling agent; and
(xiii) any mixtures thereof.

18. The method of claim 17, wherein the improvement in aesthetic appearance includes at least one of the following:
a. Decreasing skin fragility;
b. Preventing and reversing loss of collagen;
c. Preventing skin atrophy;
d. Improving skin firmness/plumpness;
e. Improving skin tone;
f. Enhancing skin thickness; and
g. Decreasing pore size.

19. The method of claim 14, wherein the improvement in aesthetic appearance includes at least one of the following:
a. Reducing dermatological aging, particularly dermatological aging due to hormonal aging;
b. Decreasing skin fragility;
c. Preventing and reversing loss of collagen;
d. Preventing skin atrophy;
e. Promoting/accelerating cell turnover
f. Improving skin firmness/plumpness;
g. Improving skin texture;
h. Decreasing fine lines and wrinkles;
i. Improving skin tone;
j. Enhancing skin thickness;
k. Decreasing pore size.
l. Minimizing skin discoloration;
m. Restoring skin luster;
n. Minimizing signs of fatigue; and
o. Reducing acne.

20. The method of claim 19, wherein the improvement in aesthetic appearance includes at least one of the following:
a. Decreasing skin fragility;
b. Preventing skin atrophy;
c. Improving skin firmness/plumpness; and
d. Enhancing skin thickness.

21. The method of claim 19, wherein the vehicle is selected from the group consisting of a solid, solution, essence, serum, pencil, spray, lotion, emulsion, cream, micro-emulsion, gel, ointment, patch, tape, and powder.

22. A topical composition comprising:
a. a phytoestrogen;
b. a vehicle; and
c. at least one secondary component selected from the group consisting of:
(i) an estrogen synthetase stimulating compound;
(ii) a 5 alpha-reductase activity inhibiting compound;
(iii) an exfoliation-promoting compound;
(iv) an ultraviolet (UV) light protecting/sunscreen agent;
(v) a retinoid;
(vi) a hirsutism inhibiting agent;
(vii) a barrier function enhancing agent;
(viii) a collagen enhancing agent;
(ix) an elastase inhibitor,
(x) a skin lightening agent
(xi) an antioxidant;
(xii) a skin cooling agent; and
(xiii) any mixtures thereof.

23. The topical composition of claim 22, wherein the at least one secondary component is selected from the group consisting of:
(i) an exfoliation-promoting compound;
(ii) an ultraviolet (UV) light protecting/sunscreen agent;
(iii) a retinoid;
(iv) a skin lightening agent
(v) an antioxidant;
(vi) a skin cooling agent; and
(vii) any mixtures thereof.

24. The topical composition of claim 22, wherein the at least one secondary component is selected from the group consisting of:
A topical composition comprising:
(i) an exfoliation-promoting compound;
(ii) an ultraviolet (UV) light protecting/sunscreen agent;
(iii) a retinoid; and
(iv) any mixtures thereof.

25. The topical composition of claim 22, wherein the at least one secondary component is selected from the group consisting of:
(i) an exfoliation-promoting compound;
(ii) an ultraviolet (UV) light protecting/sunscreen agent;
(iii) a skin lightening agent; and
(vi) any mixtures thereof.

26. The topical composition of claim 22, wherein the at least one secondary component is selected from the group consisting of:
(i) an ultraviolet (UV) light protecting/sunscreen agent.
(ii) a retinoid;
(iii) a skin lightening agent; and
(iv) any mixtures thereof.

27. The topical composition of claim 22, wherein the at least one secondary component is selected from the group consisting of:
(i) an ultraviolet (UV) light protecting/sunscreen agent;
(ii) a retinoid;
(iii) a collagen enhancing agent; and
(iv) any mixtures thereof.
